SEQUENCE LISTING (53 sequences)

SEQ ID NO: 1 - DNA, 19 bp, Artificial Sequence
  Description of Artificial Sequence Synthetic primer
gaaatttgtacagaaatgg

SEQ ID NO: 2 - DNA, 17 bp, Artificial Sequence
  Description of Artificial Sequence Synthetic primer
cttctaaatgtgtacac

SEQ ID NO: 3 - DNA, 24 bp, Artificial Sequence
  Description of Artificial Sequence Synthetic oligonucleotide
caccggagacgtgtacacgtctct

SEQ ID NO: 4 - DNA, 24 bp, Artificial Sequence
  Description of Artificial Sequence Synthetic oligonucleotide
aaacagagacgtgtacacgtctcc

SEQ ID NO: 5 - DNA, 34 bp, Artificial Sequence
  Description of Artificial Sequence Synthetic oligonucleotide
taatgggcgggacgttaacggggcggaacggtac

SEQ ID NO: 6 - DNA, 32 bp, Artificial Sequence
  Description of Artificial Sequence Synthetic oligonucleotide
cgttccgccccgttaacgtcccgcccattaat

SEQ ID NO: 7 - DNA, 25 bp, Artificial Sequence
  Description of Artificial Sequence Synthetic oligonucleotide
caccggggcgaggagctgttcaccg

SEQ ID NO: 8 - DNA, 25 bp, Artificial Sequence
  Description of Artificial Sequence Synthetic oligonucleotide
aaaccggtgaacagctcctcgcccc

SEQ ID NO: 9 - DNA, 25 bp, Artificial Sequence
  Description of Artificial Sequence Synthetic oligonucleotide
caccgggagcgcaccatcttcttca

SEQ ID NO: 10 - DNA, 25 bp, Artificial Sequence
  Description of Artificial Sequence Synthetic oligonucleotide
caccgggtgaaccgcatcgagctga

SEQ ID NO: 11 - DNA, 25 bp, Artificial Sequence
  Description of Artificial Sequence Synthetic oligonucleotide
aaactgagctcgatgcggttcaccc

SEQ ID NO: 12 - DNA, 25 bp, Artificial Sequence
  Description of Artificial Sequence Synthetic oligonucleotide
caccgtaatcggcttagaccaggac

SEQ ID NO: 13 - DNA, 25 bp, Artificial Sequence
  Description of Artificial Sequence Synthetic oligonucleotide
aaacgtcctggtctaagccgattac

SEQ ID NO: 14 - DNA, 21 bp, Artificial Sequence
  Description of Artificial Sequence Synthetic primer
caagtctccaccccattgacg

SEQ ID NO: 15 - DNA, 20 bp, Artificial Sequence
  Description of Artificial Sequence Synthetic primer
gaactccagcaggaccatgt

SEQ ID NO: 16 - DNA, 20 bp, Artificial Sequence
  Description of Artificial Sequence Synthetic primer
tccctttggtcctcttgttg

SEQ ID NO: 17 - DNA, 20 bp, Artificial Sequence
  Description of Artificial Sequence Synthetic primer
cgcccatctctgctctctac

SEQ ID NO: 18 - DNA, 20 bp, Artificial Sequence
  Description of Artificial Sequence Synthetic primer
atgctactgaggaccccatc

SEQ ID NO: 19 - DNA, 20 bp, Artificial Sequence
  Description of Artificial Sequence Synthetic primer
gcagccttgcttttctgtct

SEQ ID NO: 20 - DNA, 20 bp, Artificial Sequence
  Description of Artificial Sequence Synthetic primer
ggaggctgaggtaggaggat

SEQ ID NO: 21 - DNA, 20 bp, Artificial Sequence
  Description of Artificial Sequence Synthetic primer
ctgggactataggcgctcac

SEQ ID NO: 22 - DNA, 20 bp, Artificial Sequence
  Description of Artificial Sequence Synthetic primer
tctggtggagtttgctgttg

SEQ ID NO: 23 - DNA, 20 bp, Artificial Sequence
  Description of Artificial Sequence Synthetic primer
ccagctcatgaggttgttga

SEQ ID NO: 24 - DNA, 20 bp, Artificial Sequence
  Description of Artificial Sequence Synthetic primer
ctgaggacggaggagacaag

SEQ ID NO: 25 - DNA, 20 bp, Artificial Sequence
  Description of Artificial Sequence Synthetic primer
cgggagaggagagaggaaat

SEQ ID NO: 26 - DNA, 17 bp, Artificial Sequence
  Description of Artificial Sequence Synthetic primer
gcaacagacatacaaac

SEQ ID NO: 27 - DNA, 23 bp, Artificial Sequence
  Description of Artificial Sequence Synthetic primer
aaaactgcaaactacccaagaaa

SEQ ID NO: 28 - DNA, 19 bp, Artificial Sequence
  Description of Artificial Sequence Synthetic primer
aatctgccaccacaccttc

SEQ ID NO: 29 - DNA, 20 bp, Artificial Sequence
  Description of Artificial Sequence Synthetic primer
ggggtgttgaaggtctcaaa

SEQ ID NO: 30 - DNA, 39 bp, Artificial Sequence
  Description of Artificial Sequence Synthetic oligonucleotide
atggtgagcaagggcgaggagctgttcaccggggtggtg

SEQ ID NO: 31 - DNA, 32 bp, Artificial Sequence
  Description of Artificial Sequence Synthetic oligonucleotide
atggtgagcaagggcgaggagctgttgtggtg

SEQ ID NO: 32 - DNA, 29 bp, Artificial Sequence
  Description of Artificial Sequence Synthetic oligonucleotide
atggtgagcaagggcgaggcggggtggtg

SEQ ID NO: 33 - DNA, 41 bp, Artificial Sequence
  Description of Artificial Sequence Synthetic oligonucleotide
atggtgagcaagggcgaggagctgttcaccccggggtggtg

SEQ ID NO: 34 - DNA, 25 bp, Artificial Sequence
  Description of Artificial Sequence Synthetic oligonucleotide
atggtgagcaagggcgaggagctgt

SEQ ID NO: 35 - DNA, 42 bp, Artificial Sequence
  Description of Artificial Sequence Synthetic oligonucleotide
atggtgagcaagggcgaggagctgttcaccgggcgggtggtg

SEQ ID NO: 36 - DNA, 21 bp, Artificial Sequence
  Description of Artificial Sequence Synthetic oligonucleotide
atggtgagcaagggcgaggag

SEQ ID NO: 37 - DNA, 19 bp, Artificial Sequence
  Description of Artificial Sequence Synthetic oligonucleotide
atggtgagcaaggatggtg

SEQ ID NO: 38 - DNA, 41 bp, Artificial Sequence; identical to SEQ ID NO: 33
  Description of Artificial Sequence Synthetic oligonucleotide

SEQ ID NO: 39 - DNA, 42 bp, Artificial Sequence
  Description of Artificial Sequence Synthetic oligonucleotide
atggtgagcaagggcgaggagctgttcaccgggggggtggtg

SEQ ID NO: 40 - DNA, 38 bp, Artificial Sequence
  Description of Artificial Sequence Synthetic oligonucleotide
atggtgagcaagggcgaggagctgttcaccgggtggtg

SEQ ID NO: 41 - DNA, 44 bp, Artificial Sequence
  Description of Artificial Sequence Synthetic oligonucleotide
atggtgagcaagggcgaggagctgttcacccccggggggtggtg

SEQ ID NO: 42 - DNA, 21 bp, Artificial Sequence
  Description of Artificial Sequence Synthetic oligonucleotide
atggtgagcaagggcgaggtg

SEQ ID NO: 43 - DNA, 20 bp, Artificial Sequence
  Description of Artificial Sequence Synthetic oligonucleotide
atggtgagcaaggggtggtg

SEQ ID NO: 44 - DNA, 14 bp, Artificial Sequence
  Description of Artificial Sequence Synthetic oligonucleotide
atggtgagcaaggg

SEQ ID NO: 45 - DNA, 10 bp, Artificial Sequence
  Description of Artificial Sequence Synthetic oligonucleotide
atgggtggtg

SEQ ID NO: 46 - DNA, 29 bp, Artificial Sequence
  Description of Artificial Sequence Synthetic oligonucleotide
atggtgagcaagggcgaggagctgtggtg

SEQ ID NO: 47 - DNA, 52 bp, Artificial Sequence
  Description of Artificial Sequence Synthetic oligonucleotide
cattccgaggcatgtctgaagaggaggtgttcaccgaggtggccaacctctt

SEQ ID NO: 48 - DNA, 10 bp, Artificial Sequence
  Description of Artificial Sequence Synthetic oligonucleotide
ccaacctctt

SEQ ID NO: 49 - DNA, 47 bp, Artificial Sequence
  Description of Artificial Sequence Synthetic oligonucleotide
cattccgaggcatgtctgaagaggaggtgttcagaggccaacctctt

SEQ ID NO: 50 - DNA, 54 bp, Artificial Sequence
  Description of Artificial Sequence Synthetic oligonucleotide
cattccgaggcatgtctgaagaggaggtgttcaccgccaggtggccaacctctt

SEQ ID NO: 51 - DNA, 27 bp, Artificial Sequence
  Description of Artificial Sequence Synthetic oligonucleotide
cattattattattattattattattga

SEQ ID NO: 52 - DNA, 14 bp, Artificial Sequence
  Description of Artificial Sequence Synthetic oligonucleotide
ctttgtaattatat

SEQ ID NO: 53 - DNA, 13 bp, Artificial Sequence
  Description of Artificial Sequence Synthetic oligonucleotide
aggtcccagctgt

(19) United States
(12) Patent Application Publication
(10) Pub. No.: US 2017/0145438 A1
(43) Pub. Date: May 25, 2017

(54) Viral Vectors for Gene Editing

(71) Applicant: University of South Carolina, Columbia, SC (US)

(72) Inventor: Boris Kantor, Chapel Hill, NC (US)

(21) Appl. No.: 15/347,996

(22) Filed: Nov. 10, 2016

Related U.S. Application Data
(60) Provisional application No. 62/259,362, filed on Nov. 24, 2015.

(51) Int. Cl. C12N 15/86; C12N 9/22; C12N 7/00

(52) U.S. Cl. CPC C12N 15/86; C12N 7/00; C12N 9/22; C12N 2740/10043; C12N 2740/15043; C12N 2740/16043; C12N 2830/34

(57) ABSTRACT

Disclosed are delivery platforms for use in gene editing that include a relatively short, highly efficient promoter that drives transcription of a nucleic acid sequence that encodes a gene-editing molecule, e.g., either a gRNA or a nuclease. In conjunction with this promoter, the vector includes one or more transcription factor binding elements (an Sp1 binding element and/or an NF-κB binding element) cloned into the vector upstream of a promoter that drives transcription of a gene-editing molecule. The vector can be a all-in-one CRISPR/Cas9 delivery platform and can incorporate one or more of the transcription factor binding elements upstream of a promoter for the gRNA component and/or of a promoter for the nuclease component.

### CROSS REFERENCE TO RELATED APPLICATION

This application claims filing benefit of U.S. Provisional Patent Application Ser. No. 62/259,362, having a filing date of Nov. 24, 2015, which is incorporated herein by reference in its entirety.

### SEQUENCE LISTING

The instant application contains a Sequence Listing which has been filed electronically in ASCII format and is hereby incorporated by reference in its entirety. Said ASCII copy, created on Oct. 31, 2016, is named USC-499(1175)_SL.txt and is 12,529 bytes in size.

### BACKGROUND

The ability to alter the function of a targeted gene through genome editing is desirable for both research and therapeutic perspectives. The recent discovery of clustered regularly interspaced short palindromic repeats (CRISPR) and CRISPR-associated (e.g., CRISPR/Cas9) systems has revolutionized the field of genome editing by providing unprecedented control over gene expression in many species, including humans. Integrase-competent lentiviral vectors (ICLVs) are one of the mainstays of current delivery platforms for gene editing systems such as the CRISPR/Cas9-based system, which combines a Cas9 nuclease in conjunction with guide RNA (gRNA) having complementarity to the DNA target site. ICLV-based systems have shown low immunogenicity, ability to accommodate large DNA payloads, and efficient transduction of a wide range of dividing and non-dividing cells.

ICLV-based CRISPR/Cas9 systems have been successfully exploited for modeling cancer in mice and in many preclinical applications including targeting of HIV-1, hepatitis B virus (HBV), and HSV-1 infections as well as genetic correction in diseases such as Tyrosinemia and Cystic Fibrosis. Notwithstanding these successes, integrating platforms may not be ideal for therapeutic delivery of gene editing materials such as CRISPR RNA-guided nucleases (RGNs), as their overexpression may come at a price of undesired side effects including promiscuous interactions of excess gRNA/Cas9 with off-target genes and risk of insertional mutagenesis.

Studies supporting such concerns indicate a need for transient methods of vector-mediated delivery and have driven the development of non-integrating systems. For instance, integrase-deficient lentiviral vectors (IDLVs) have been used to deliver transgenes to a number of target organs, including the eye, liver, brain, muscle, and lymph nodes; and have proven effective in correcting Leber congenital amaurosis disease, and factor IX (FIX) hemophilia B in mice. The ability of IDLVs to transduce cells efficiently and transiently deliver payloads makes them an attractive non-integrating gene-delivery platform for the expression of genome-editing systems such as the CRISP R/Cas9 system. Transient delivery vector systems have been found to be highly advantageous in preclinical and clinical settings where short-term expression of potentially genotoxic gene-editing transgenes is required. Non-integrating vectors have also been successfully employed as a means of avoiding genotoxicity associated with continuous expression of transgenes, e.g., zing-finger nucleases (ZFNs), as well as in delivery of the donor DNA template required for DNA repair-mediated gene editing in vitro. These and other proof-of-concept studies provide a solid foundation for further exploration and development of non-integrating platforms for gene-editing applications.

While improvements have been made in both integrating and non-integrating gene-editing viral platforms, issues still exist. For instance, one shortcoming of all-in-one integrating lentiviral vector systems used for the delivery of CRISPR/Cas9-based materials is low production titers. Methods to overcome such problems have included development of binary-plasmid vector systems in which the Cas9 and gRNA components are delivered separately. This approach has improved production yields, but is not suitable for gene-editing applications including in-vivo screening and disease-modeling. Second generation integrating all-in-one vectors have shown increase in production titer and transduction efficiency over the first-generation systems, but these are still about 25-fold lower production yields compared with traditional vectors.

Moreover, the significantly reduced levels of transgene expression in non-integrating systems as compared to integrating systems remains a key issue in developing clinically effective non-integrating gene editing vectors. The low expression of integrase-deficient vectors has been linked to the formation of closed-chromatin structure around episomal DNA and demonstrated to be enriched with post-translational histone modifications typical for the negatively-regulated, silencing genes. The removal of these negative elements embedded into the expression cassette has been demonstrated to be an efficient strategy for enhancing an expression of the episomal vectors. For instance, removal of cis-acting sequences within the U3 region of the IDLV's LTR has been shown to improve episomal transgene expression by nearly 3-fold as demonstrated by measuring an expression of GFP by flow cytometry method, and by more than 10-fold by measuring Luciferase expression by Luciferase-assay. However, other mechanisms of episomal inhibition may be involved because expression remains below that of similar integrating systems.

Another approach to improving expression levels has been the inhibition of cellular restriction factors. Applying this approach in conjunction with IDLV preparations has improved episomal expression to levels observed with normal integrating ICLVs, but as mentioned, integrating systems also exhibit less than desirable expression levels. Other approaches for improving vector production titers and transgene expression include trans-targeting of negative chromatin factors with small molecules in the forms of deacetylase- and proteasome-inhibitors, as well as stabilizing RNA and proteins via blocking exoribonuclease activities and the codon optimization, respectively.

While the above describe improvements in the art, room for further improvement exists. What are needed in the art are vector systems that can be utilized for highly targeted gene editing and that can provide improved transgene expression. Systems that can provide high production titers with low off-target interaction would be of great benefit.

### SUMMARY

According to one embodiment, disclosed is a viral vector for use in gene editing applications. The viral vector can include one or more structural components derived from a virus (e.g., lentiviral vector components), and a nucleic acid sequence that encodes a gene-editing molecule, e.g., a gRNA or a nuclease. In addition, the viral vector can include a promoter configured to initiate transcription of the nucleic acid sequence. More specifically, the promoter can be a promoter that is free of internal promoter sequences such as an RNA polymerase III promoter or a modified RNA polymerase II promoter. The viral vector also includes an Sp1 transcription factor binding element or an NF-κB transcription factor binding element upstream of the promoter and, in one embodiment, immediately upstream of the promoter. In one embodiment, the viral vector can be an all-in-one plasmid vector such as a CRISPR/Cas9 vector and the vector can include an Sp1 and/or an NF-κB transcription factor binding element immediately upstream of either or both of the promoter for the gRNA segment and the promoter for the nuclease segment. Beneficially, a vector as disclosed herein can be integrating (e.g., an ICLV) or non-integrating (e.g., an IDLV) and in either case can provide for high production titers.

According to another embodiment, disclosed is a delivery platform that can include a vector plasmid for gene editing (e.g., lentiviral or other) in conjunction with additional plasmids. For instance, the vector plasmid can include an Sp1 and/or NF-κB transcription factor binding element upstream of a promoter for a gene-editing molecule as described. Other plasmids included in the platform can include a packaging plasmid of lentivirus, a rev expression plasmid, an envelope plasmid, or packaging plasmids necessary for the assembly of other vector systems (adeno-associated vector, retroviral vector, etc.).

### BRIEF DESCRIPTION OF THE FIGURES

The present disclosure may be better understood with reference to the figures including:

FIG. 1 presents a map of the lentiviral vector cassette plasmid, pBK176 harboring two copies of Sp1 binding sites (marked with arrows). Other regulatory elements of the pBK176 plasmid includes, primer binding site (PBS), splice donor (SD) and splice acceptor (SA), central polypurine tract (cPPT) and polypurine tract (PPT), Rev Response element (RRE), Woodchuck Hepatitis Virus Posttranscriptional Regulatory Element (WPRE), and the retroviral vector packaging element, psi (Ψ) signal. A human Cytomegalovirus (hCMV) promoter, a core-elongation factor 1α promoter (EFS), and a human U6 promoter are also included. The self-inactivated vector (SIN) cassette plasmid carries a deletion (−18 bps to −418 bps) in the U3 region of 3′-LTR (ΔU3). A polylinker site contains a pair of BsmBI sites and a unique BsrGI site used for cloning of sgRNA and for its verification, respectively.

FIG. 2 presents a map of the lentiviral vector cassette plasmid, pBK109 from which pBK176 of FIG. 1 was derived. The pBK109 cassette includes the same elements as the pBK176 cassette save for the two copies of the SP1 binding sites.

FIG. 3 presents production titers of integrating (ICLV) and non-integrating (IDLV) viral particles vBK198 and vBK109 including the plasmids of FIG. 1 (pBK176) and FIG. 2 (pBK109), respectively, packaged into the viral particles. The results are recorded in copy number per milliliter, equating 1 ng of p24gag to 1×10⁴ particles.

FIG. 4 presents the results of functional viral titers for integrase-competent vBK198 that includes the pBK176 plasmid of FIG. 1 and f vBK109 that includes the pBK109 plasmid of FIG. 2 as determined by screening and counting puromycin-resistant colonies. Results are recorded as a ratio between the two virus types.

FIG. 5 illustrates the efficiency of CRISPR/Cas9 mediated knockout. The level of eGFP depletion was evaluated for sgRNAs (1-3) delivered by ICLV by fluorescence-activated cell sorting assay 7 days pt. Naïve HEK293T-eGFP cells and non-sgRNA-expressed cells are presented as controls.

FIG. 6 illustrates the efficiency of eGFP-sgRNA1/Cas9 packaged into integrating and nonintegrating viral particles transduced into HEK293T-eGFP cells. The levels of eGFP depletion were evaluated by fluorescence-activated cell sorting assay at days 7 (top row), 14 (middle row) and 21 (bottom row) pt. The percentage of eGFP-positive cells remaining after transduction was recorded. No-virus and non-sgRNA cells are presented as controls.

FIG. 7 illustrates the integration rates as determined via isolation of gDNA from HEK293T-eGFP cells transduced by IDLV-CRISPR/Cas9 at days 1, 3, 7, 14 and 21 pt following qPCR analysis. The results are recorded as a ratio between copy numbers per cell calculated for each time point to that of day 1.

FIG. 8 illustrates the integration rates as determined via isolation of gDNA from HEK293T-eGFP cells transduced by ICLV-CRISPR/Cas9 at days 1, 3, 7, 14 and 21 pt following qPCR analysis. The results are recorded as a ratio between copy numbers per cell calculated for each time point to that of day 1.

FIG. 9 presents an evaluation of the efficiency and the specificity of IDLV-CRISPR/Cas9 and ICLV-CRISPR/Cas9 systems. T7 endonuclease I assay was performed on HEK293T-eGFP cells transduced by IDLV-sgRNA1/Cas9 or ICLV-sgRNA1/Cas9 at days 7 and 21 pt. Results shown include results of on-target (eGFP) specificity evaluated at days 7 pt (top, left panel) and 21 days pt (top, right panel). The gDNA isolated from the transduced cells was amplified with eGFP-specific primers and treated with T7 endo I (+) or left untreated (−). Lane 1: naïve (untransduced cells); lane 2: ICLV-transduced cells at MOIs=1; lane 3: ICLV-transduced cells at MOIs=5; lane 4: IDLV-transduced cells at MOIs=1; lane 5: IDLV-transduced cells at MOIs=5 and lane 6: ICLV-transduced cells non-sgRNA-control. Also shown are the results of analyzing InDels formation by Sanger sequencing. ICLV-transduced cells (bottom, left) or IDLV-transduced cells (bottom, right) at MOIs=1 were used to evaluate rate of InDels formation at day 7 pt. The unmodified GFP-target sequence is underlined in bold. The analysis of 10-clones (out of 50) is illustrated. Formed insertions and deletions are in bold, and by dropped line, respectively. FIG. 9 discloses SEQ ID NOS 30-46, respectively, in order of column.

FIG. 10 presents the results of determination of off-target effects following IDLV-CRISPR/Cas9 and ICLV-CRISPR/Cas9 transduction. The InDels frequencies were evaluated for Sin3B gene at days 7 pt (top) and 21 pt (middle). The arrow heads that point to two bands at lane 3 of the 21 day panel are underscore off-target cuts in Sin3B gene (bottom). Sanger sequencing analysis of Sin3B-amplified DNA following ICLV-transduction at day 21 pt. Four clones (out of 50) shown InDels formation are illustrated. FIG. 10 discloses SEQ ID NOS 47-50, respectively, in order of appearance.

FIG. 11 presents off-target effects within MTRF-1L (top left), NARF (bottom left), INSC-1 (top right) and BRAF-1 (bottom right) genes analyzed at day 21 after transduction with ICLV-CRISPR/Cas9 using T7 endonuclease I assay. Lane 1: Non-sgRNA control; lane 2: ICLV-transduced cells at MOI=1; lane 3: ICLV-transduced cells at MOI=5; lane 4: IDLV-transduced cells at MOI=5.

FIG. 12 presents evaluations of IDLV-CRISPR/Cas9 and ICLV-CRISPR/Cas9 off-target effects. Included is the average of InDels (%) calculated for ten off-target genes detected by WES of IDLV-CRISPR/Cas9 and IDLV-CRISPR/Cas9-samples (top left). Genes harboring InDels are shown in the graph at the bottom left. The rates of InDels for IDLV (light bar) or ICLV (dark bar) are calculated as a ratio (%) between reads with mutated sequences and total reads. Genes harboring InDels are highlighted (top right) and insertions (CELA3A (SEQ ID NO: 51), NA, NET1 (SEQ ID NO: 52), IFITM1, SYT8, ROBO4 (SEQ ID NO: 53), CGV1, LAMAS, CHRNA4) and deletions (ACCN2) are underlined. A genomic position (chromosome number) and targeted-DNA strand (+ or − strand) are also shown (top right).

FIG. 13 presents the results of an evaluation of the ability of IDLV-CRIPSR/Cas9 to target GABA α2 receptor knockout in rat brain. The depletion of GABAA α2 receptor expression was estimated by Western blot analysis. The level of protein expression was evaluated for the injected ventral hippocampal area (upper panel, lanes 3&4) and naïve (untreated) dorsal hippocampal area (upper panel, lanes 1&2). Tubulin (DM1A) antibody was used as a loading control.

### DETAILED DESCRIPTION

Reference will now be made in detail to various embodiments of the disclosure, one or more examples of which are illustrated in the accompanying drawings. Each example is provided by way of explanation of the subject matter, not limitation thereof. In fact, it will be apparent to those skilled in the art that various modifications and variations can be made in the present disclosure without departing from the scope or spirit of the subject matter. For instance, features illustrated or described as part of one embodiment can be used in another embodiment to yield a still further embodiment.

The present disclosure generally relates to a delivery platform for use in gene editing. More specifically, the delivery platform can include a relatively short, highly efficient promoter that drives transcription of a nucleic acid sequence that encodes a gene-editing molecule, e.g., either a gRNA or a nuclease. In conjunction with this promoter, the vector includes one or more transcription factor binding elements. More specifically, the transcription factor binding element(s) can be cloned into the vector upstream (e.g., immediately upstream) of a promoter that drives transcription of a gene-editing molecule. The transcription factor binding element can include an Sp1 binding element and/or an NF-κB binding element. In one embodiment, the vector can be an all-in one CRISPR/Cas9 delivery platform and can incorporate one or more of the transcription factor binding elements upstream of a promoter for the gRNA component and/or of a promoter for the nuclease component.

The delivery platform can be either non-integrating or integrating. For example, in one embodiment a non-integrating IDLV vector is disclosed having genome-wide targeting specificity that can, in contrast to traditional integrating systems, provide high efficiency of transient delivery of gene-editing molecules without formation of off-target InDels.

Introduction of a binding element upstream of a promoter for a gene-editing sequence can dramatically improve transcription efficiency. For instance, the disclosed vectors can exhibit high efficiency in mediating rapid gene knockouts in cells including both dividing cells as well as in non-dividing cells, e.g., brain neurons.

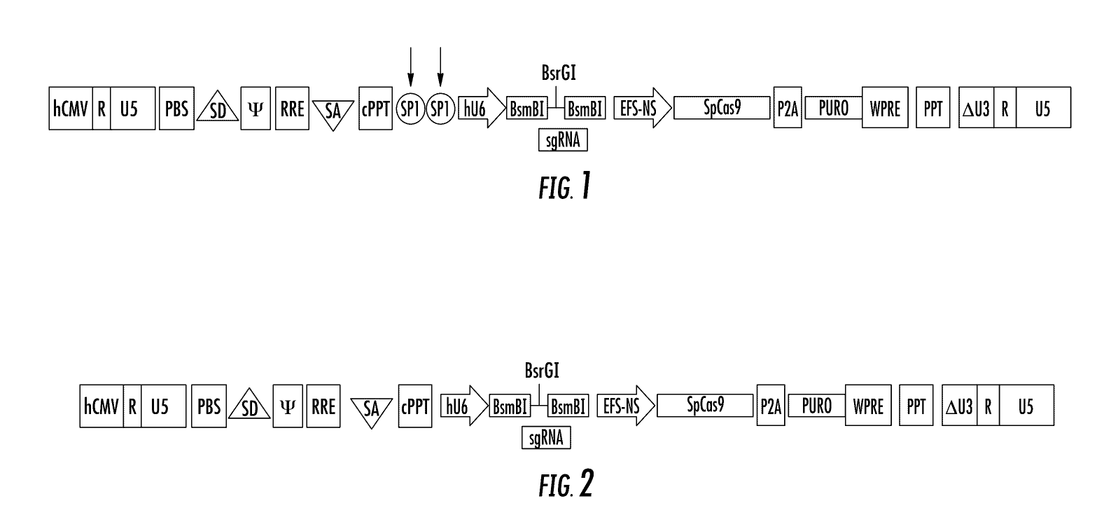

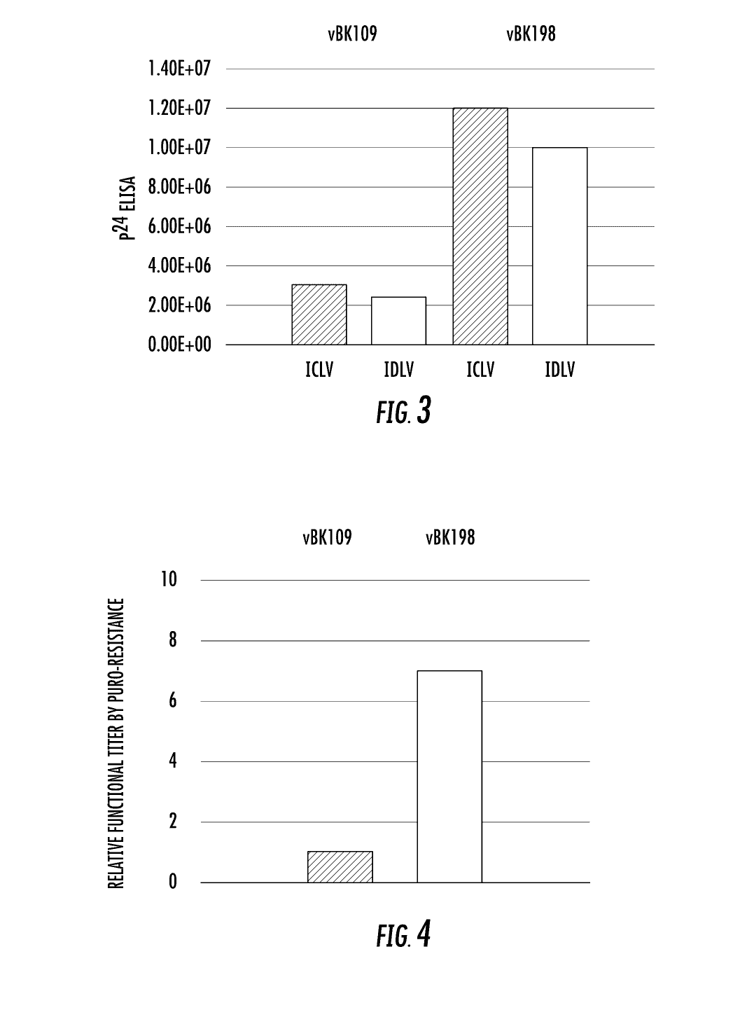

FIG. 1 illustrates one embodiment of a viral vector plasmid encompassed herein. The vector of FIG. 1 is an all-in-one CRISPR/Cas9 lentiviral vector plasmid similar to the more traditional vector plasmid of FIG. 2. However, the vector of FIG. 1 has been modified to include two Sp1 transcription factor binding elements (designated by the arrows) upstream of a hU6 promoter that drives transcription of the sgRNA nucleotide.

A vector can include one or more Sp1 binding elements as illustrated in FIG. 1 or alternatively, can include one or more NF-κB binding elements in this locale. In addition, a vector can be modified to include both Sp1 binding element(s) and NF-κB binding element(s) upstream of a promoter for a gene-editing component.

The Sp1 transcription factor contains a zinc finger protein motif, by which it binds directly to DNA and enhances gene transcription. The NF-κB factor is known to directly bind DNA and is involved in DNA transcription control. NF-κB belongs to the category of “rapid-acting” primary transcription factors, i.e., transcription factors that are present in cells in an inactive state and do not require new protein synthesis in order to become activated. Previous studies have demonstrated that transcription factors Sp1 and NF-κB are important for efficient production and replication of the wild type HIV-1. For instance, Berkhout, et al. (J Virol. 1999 February; 73(2); 1138-1145) demonstrated that live, attenuated HIV-1 can gain virulence via duplication of the region encoding binding sites for the Sp1 transcription factor, and Bachu, et al. (2012 December 28; 287(53)) demonstrated that NF-κB binding sites in HIV-1 subtype C LTR confer selective advantage and increased infectious capability. In formation of viral vectors, however, regions harboring the Sp1 binding sites and NF-κB binding sites have been deleted for safety reasons.

According to the present disclosure, reintroduction of Sp1 and/or NF-κB transcription factor binding site(s) to a vector cassette upstream of a promoter for a nucleotide encoding a gene-editing molecule can enhance production efficacy of the vector system without surrendering the safety features gained by wild-type regional deletions. For example, inclusion of one or more Sp1-binding sites upstream of a promoter can result in an approximately eightfold increase of transduction efficiency of both integrating and non-integrating CRISPR/Cas9 vectors compared to their second-generation counterparts.

Expression of gRNAs and nucleases having correct sequences is a critical step for successful gene-editing technologies. For example, for gRNAs of the CRISPR system, the first approximately 20 nucleotide sequence of the gRNA transcript defines the CRISPR target. Thus, precision at the 5′ end of these small RNA transcripts is important for these technologies to work properly. As such, in formation of the disclosed vectors, promoters that can be associated with the reintroduced transcription factor binding element(s) and the gene-editing components can include relatively short promoters capable of providing a well-defined transcription initiation site for a gene-editing component of the vector. Promoters capable of such well-defined transcription initiation sites include relatively short promoters (e.g., about 300 base pairs or less, or about 200 base pairs or less in some embodiments). In one embodiment, the promoters associated with the reintroduced transcription factor binding sites can be free of internal promoter sequences (i.e., introns). By way of example and without limitation, these promoters of the disclosed vectors can include RNA polymerase III promoters and RNA polymerase II promoters that are either naturally free of internal promoter sequences or have been modified to remove internal promoter sequences.

As utilized herein, “RNA polymerase III promoter” or “RNA pol III promoter” or “polymerase III promoter” or “pol III promoter” is meant any invertebrate, vertebrate, or mammalian promoter, e.g., human, murine, porcine, bovine, primate, simian, etc. that, in its native context in a cell, associates or interacts with RNA polymerase III to transcribe its operably linked gene, or any variant thereof, natural or engineered, that will interact in a selected host cell with an RNA polymerase III to transcribe an operably linked nucleic acid sequence.

As utilized herein, “RNA polymerase II promoter” or “RNA pol 11 promoter” or “polymerase II promoter” or “pol 11 promoter” is meant any invertebrate, vertebrate, or mammalian promoter, e.g., human, murine, porcine, bovine, primate, simian, etc. that, in its native context in a cell, associates or interacts with RNA polymerase II to transcribe its operably linked gene, or any variant thereof, natural or engineered, that will interact in a selected host cell with an RNA polymerase II to transcribe an operably linked nucleic acid sequence.

A vector can include the Type III RNA pol III promoters including, but not limited to, U6, H1, MRP, and 7SK promoters that exist in the 5′ flanking region, include TATA boxes, and lack internal promoter sequences. Such promoters are known in the art and can be obtained by searching public sequence databases such as GenBank®. Variant forms, i.e., copies, of these promoters may be utilized and may function equally or more effectively. For example, alternative, synthetic variant forms of a pol III promoter can include truncated or extended lengths and/or nucleotide substitutions with respect to the canonical promoter, as is known.

Pol III promoters for utilization in an expression construct to transcribe a gene-editing molecule may advantageously be selected for optimal binding and transcription by the host cell RNA polymerase III, e.g., utilizing human or other mammalian pol III promoters in an expression construct designed to transcribe nucleotides encoding gene-editing molecules in human host cells. For applications involving expression by an endogenous RNA III polymerase in a non-mammalian host cell, e.g., in an avian, fish, or invertebrate host cell, it may be advantageous to select cognate RNA pol III promoters, e.g., avian, fish, etc. promoters.

Without wishing to be bound to any particular theory, it is believed that one reason RNA Pol III promoters are useful for expression of small engineered RNA transcripts is that RNA Pol III termination occurs efficiently and precisely at a short run of thymine residues in the DNA coding strand, without other protein factors, T₄ and T₅ being the shortest Pol III termination signals in yeast and mammals, with oligo (dT) terminators longer than T₅ being very rare in mammals. Accordingly, the polymerase III promoter expression construct can include an appropriate oligo (dT) termination signal, i.e., a sequence of 4, 5, 6 or more Ts, operably linked 3′ to each RNA Pol III promoter in the DNA coding strand. A DNA sequence encoding an engineered RNA, e.g., a gRNA to be transcribed, may then be inserted between the Pol III promoter and the termination signal.

In one embodiment, a promoter associated with a transcription factor binding element and a nucleotide encoding a gene-editing molecule can be a Pol II promoter that has been modified to remove internal elements. For example a vector can include at this locale the short form of the elongation factor—1α promoter (EFS), a tetracycline responsive element-minimal CMV promoter, a modified CBA promoter (CBh) or others.

The promoter associated with the upstream transcription factor binding element(s) can drive transcription of a nucleotide sequence that encodes a gene-editing molecule, e.g., a gene-editing nuclease or a gRNA. Any gene editing nuclease as is known in the art can be encoded in the gene-editing component of the vector including, without limitation, zinc finger nucleases (ZFNs), Transcription Activator-Like Effector Nucleases (TALENs), nucleases of the CRISPR/Cas system, and engineered meganuclease re-engineered homing endonucleases.

In one embodiment, the vector can be a CRISPR/Cas vector and the promoter associated with the upstream transcription factor binding element can drive transcription of either or both of a gRNA and/or a gene-editing nuclease of the system. Natural CRISPR/Cas systems are used by various bacteria and archaea to defend against viruses and other foreign nucleic acids. Recent publications have shown that Type II CRISPR/Cas systems can be engineered to direct double-stranded DNA breaks (DSBs) in vitro to specific sequences by using guide RNA (gRNA) with complementarity to DNA target site and Cas9 nuclease. The adaptation of this system for gene editing has had a tremendous impact on development of disease models in animals, identification and validation of novel therapeutic targets, and correction of genetic mutations in humans.

Referring again to FIG. 1, in this embodiment, the vector includes two Sp1 binding elements immediately upstream of a human U6 promoter. This promoter drives transcription of the gRNA component of the CRISPR/Cas system. As illustrated, the nuclease component includes an EFS-NS promoter that drives transcription of the SpCas9 nucleotide sequence. As shown, this particular vector includes the reintroduced transcription factor binding elements only at the gRNA section of the vector. It should be understood however, that when considering a multiple plasmid vector such as an all-in-one CRISPR/Cas9 vector, one or more of the promoters of the gene-editing components can be associated with an upstream transcription factor binding elements. For instance, either or both of the gRNA encoding nucleotide sequence and the nuclease encoding nucleotide sequence of an all-in-one CRISPR/Cas9 plasmid vector can include a transcription factor binding element upstream of the associated promoter.

The vector that includes the gene-editing component and associated sequences may be a viral vector such as an adenoviral vector, a retroviral viral vector (e.g., a lentiviral vector, a pBABE vector, etc.), a reoviral vector, or an adeno-associated viral vector and as such can include structural components derived from the virus upon which the vector is based. In one embodiment the vector may be derived from a virus that naturally replicates as an extrachromosomal element such as an artificial chromosome or an Epstein Barr based virus. A virus can enter a host cell via its normal mechanism of infection or can be modified such that it binds to a different host cell surface receptor or ligand to enter a cell via pseudotyping with envelopes derived from VSV, Rabies Mokola, RRV, LCMV, MuLV, Syndbis and other viruses' glucoproteins.

In one embodiment, the vector can be a retroviral vector. Natural retroviruses carry their genetic information in the form of RNA; however, once the virus infects a cell, the RNA is reverse-transcribed into the DNA form which integrates into the genomic DNA of the infected cell. The integrated DNA form is called a provirus. In one embodiment, the retroviral vector can be a lentiviral vector and can include structural components derived from a lentiviral genome or a portion thereof in combination with additional sequences. In one particular embodiment, the vector can be an HIV-1 lentiviral vector. As used herein, the term lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. A “lentiviral vector” is a type of retroviral vector well-known in the art (see, e.g., Trono D. (2002) Lentiviral vectors, New York: Spring-Verlag Berlin Heidelberg).

A lentiviral vector can be based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle can be based on a genetically or otherwise (e.g. by specific choice of packaging cell system) altered version of a particular retrovirus.

That the vector and/or vector particle is “based on” a particular virus means that the vector is derived from that particular retrovirus. The genome of the vector particle can include components from that retrovirus as a backbone. For example, the vector particle can include vector components compatible with the RNA genome, including reverse transcription and integration systems. Generally, these can include gag and pol proteins derived from the particular retrovirus. Thus, a number of the structural components of the vector can be derived from that retrovirus, although they may have been altered genetically or otherwise so as to provide desired useful properties. However, certain structural components and in particular the gene-editing components and related sequences discussed above, may originate from a different source. The vector host range and cell types infected or transduced can be altered by using different sequences in the vector particle production system that can be derived from other sources including other viruses to give the vector particle a desired structure and function.

A vector as disclosed herein can be an integrating or a non-integrating vector. For instance, in one embodiment, a vector can be derived from a typical integrating lentiviral vector as is known in the art. In another embodiment, the vector can be a non-integrating lentiviral vector that has been modified so as to inhibit the normal integration process. For example, viral integration can be inhibited via deletion or mutation of an integrase protein encoded on the vector, thereby producing an integrase-defective vectors.

Certain vectors, such as the HIV-1 vector, code lentiviral integrase by a region (e.g., a pol region) that cannot be deleted as this region encodes other critical activities such as reverse transcription, nuclear import, and viral particle assembly. Accordingly, in such as embodiment, the region can be mutated to inhibit integration of the vector. Mutations in pol that alter the integrase protein can fall into one of two classes: those which selectively affect only integrase activity (Class I); or those that have pleiotropic effects (Class II). Mutations throughout the N and C terminals and the catalytic core region of the integrase protein generate Class II mutations that affect multiple functions including particle formation and reverse transcription. Class II mutations may not be suitable when designing non-integrating lentiviral vectors, because they can disrupt functions that are critical for vector processing and expression. Class I mutations are limited in effect to catalytic activities, DNA binding, linear episome processing and multimerization of integrase. The most common Class I mutation sites, all of which are encompassed herein, are a triad of residues at the catalytic core of integrase, including D64, D116, and E152. Each of these mutations has been shown to efficiently inhibit integration with a frequency of integration up to four logs below that of normal integrating vectors while maintaining transgene expression of the vector.

In one embodiment, integration can be inhibited in a normally integrating vector via mutation in the integrase DNA attachment site (LTR att sites) within a 12 base-pair region of the U3 or an 11 base-pair region of the U5 regions at the terminal ends of the 5′ and 3′ LTRs, respectively. These sequences include the conserved terminal CA dinucleotide which is exposed following integrase-mediated end-processing. Single or double mutations at the conserved CA/TG dinucleotide can result in up to a three to four log reduction in integration frequency; however, this vector embodiment can retain all other necessary functions for efficient viral transduction.

A vector as described herein can include other components as are generally known in the art. For instance, an important safety feature of most lentiviral vectors is the inclusion of a Self-Inactivating Long Terminal Repeat (SIN-LTR). This feature can minimize the risk of producing a replication-competent lentivirus by recombination with wild-type viruses. The mechanism involves taking advantage of the normal replication cycle of the lentivirus, e.g., HIV-1. In wild-type HIV-1, the viral promoter is within the U3 region of the 5′ LTR and is required to generate the full length viral transcript. The U3 region is also present in the 3′ LTR but is not essential in the DNA form of the virus. During viral replication, the RNA genome is reverse transcribed and the 3′ LTR can be utilized in formation of both the 5′ and 3′ LTR of the daughter virus. By incorporating a large deletion into the U3 region of the 3′ LTR any progeny will contain two inactivated LTR after reverse transcription. Transgene expression can be dependent solely on the internal promoter(s) (e.g., a promoter free of internal promoter sequences as described previously).

A vector can include other components as are known in the art, e.g., any of various selection markers and/or reporter genes. Examples of reporter genes which may be employed to identify transfected cell lines include alkaline phosphatase (AP), beta galactosidase (LacZ), beta glucoronidase (GUS), chloramphenicol acetyltransferase (CAT), green fluorescent protein (GFP), horseradish peroxidase (HRP), and luciferase (Luc). Possible antibiotic selectable markers include those that confer resistance to ampicillin, bleomycin, chloramphenicol, gentamycin, hygromycin, kanamycin, lincomycin, methotrexate, phosphinothricin, puromycin, and tetracyclin.

Referring again to FIG. 1, a lentiviral vector can contain on the 5′ and 3′ ends the minimal LTR regions required for integration of the vector including for example an untranslated segment (U5/U3), a flanking repeat region (R), and a human Cytomegalovirus (hCMV) promoter. Following the 5′ LTR region can be the primer binding site (PBS), the splice donor (SD) and the psi (Ψ) signal that is required for packaging of the vector RNA into the particle. This region can be followed by the rev response element (RRE), the splice acceptor (SA), and the central polypurine tract (cPPT), which can enhance vector production by transporting the full length vector transcript out of the nucleus for efficient packaging into the vector particle. Next is the gene-editing segment, which in this embodiment includes the transcription factor binding site(s) (e.g., two Sp1 binding sites, as illustrated) upstream of the hU6 promoter, which drives transcription of the gRNA segment. The polylinker site contains a pair of BsmBI sites and a BsrGI site used for cloning of sgRNA and for its verification, respectively. A core-elongation facto r 1α promoter (EFS) can be used to drive transcription of the nucleotide sequence encoding the Cas9 protein, as shown. A vector can also include a polypurine tract (PPT), Woodchuck Hepatitis Virus Posttranscriptional Regulatory Element (WPRE), and the retroviral vector packaging element, as are known in the art. Of course, variations and modifications of the various genetic elements of the embodiment illustrated in FIG. 1 as are generally known in the art are encompassed herein.

The genetic elements can be processed to form a full length RNA molecule that can be packaged into the vector particle and can contain all of the genetic information that will be transduced into the host cells.

In addition to a transgene plasmid containing the gene-editing vector as described above, a system can include a plasmid expressing the gag and pol gene regions that produce the HIV-1 structural proteins required for capsid formation and genome integration. A plasmid expressing HIV-1 rev can also be included to activate the rev responsive element engineered into the transgene and gag/pol plasmids. This can facilitate nuclear transport and can also be included as a safety feature. A fourth plasmid can be included that can express an envelope glycoprotein that engages receptors on the target cells. As the native HIV-1 glycoprotein is generally restricted to CD4 positive cells, a system can include alternative envelopes in some embodiment, for instance a Vesicular Stomatitis Virus G glycoprotein (VSV-G), to facilitate uptake into a wide variety of species and cell types. The use of multiple plasmids and the requirement for rev can be included to minimize recombination events that can lead to the development of a replication competent virus.

The full length RNA transcript can be packaged inside a capsid of a vector particle that contains the nucleocapsid, capsid, and matrix proteins that can be generated in one embodiment from a packaging plasmid. A reverse transcriptase polymerase that can be generated from the packaging plasmid can also be located within the capsid with the RNA transcript. The capsid can thus encase and protect the full length RNA transcript.

A lentiviral vector can be generated according to standard methodology, e.g., by introducing the transgene and packaging plasmids into cells, for instance HEK293T cells as are commonly utilized. Vector supernatant can be collected from the media. The lentiviral vector particles can then be concentrated by ultracentrifugation and purified e.g., using a combination of chromatography, tangential flow filtration and diafiltration as are known in the art.

During use, the vectors can transduce a target cell. In order to effectively transduce a target cell, both integrating and non-integrating vectors (e.g., ICLV and IDLV, respectively) must retain the ability to readily enter the cell, form a pre-integration complex, be transported into the nucleus and efficiently express their gene-editing genetic payload. Depending on the envelope pseudotype used, the membrane bound particles can enter cells either by direct fusion with the plasma membrane or via a receptor-mediated endosomal pathway. In the direct fusion pathway the particle can be uncoated upon entry and release the viral contents into the cytoplasm. This can allow for reverse transcription of the viral RNA into linear cDNA and development of the pre-integration complex (PIC). The PIC includes the reverse transcribed viral cDNA complexed with integrase, matrix, reverse transcriptase, and nucleocapsid proteins. The endosomal pathway is dependent upon the pH within the endosome for membrane fusion, subsequent uncoating, and PIC formation within the cytoplasm. The transportation of the PIC to the nucleus is not completely understood, but is believed to occur by an ATP-dependent process via nucleoporins using nuclear localization signals and cellular transport mechanisms. Certain of the known localization signals can be removed during vector design; nevertheless, the transduction of quiescent cells by viral vectors is well documented.

The present invention may be better understood with reference to the Example, set forth below.

### EXAMPLE

### Materials and Methods

Plasmids Construction:

Integrase-deficient packaging cassette was derived from psPAX2 (Addgene #12260) as follows: The int region was amplified with the following primers:

F-
(SEQ ID NO: 1)
5′-GAAATTTGTACAGAAATGG-3′
R-
(SEQ ID NO: 2)
5′-CTTCTAAATGTGTACAC-3′

The R-primer harbored a T-G mutation in the GAT-codon which created a substitution of Asp (D) to Glu (E)-(D64E). The PCR product harboring the mutation was digested with BsrGI enzyme and was cloned into psPAX2 replacing the corresponding region. The int-packaging cassette was named pBK43. The presence of the mutation was confirmed by sequencing analysis. To generate pLenti-CRISPR/Cas9-expressing cassette, pLentiCRISPRv2 (Addgene, #52961) was digested with BsmBI (removing 2-kb of a buffer) and cloned with a pair of annealed and phosphorylated oligonucleotides:

upper-
(SEQ ID NO: 3)
5′-CACCGGAGACGTGTACACGTCTCT-3′
lower-
(SEQ ID NO: 4)
5′-AAACAGAGACGTGTACACGTCTCC-3′

The resulting plasmid, pBK109 (FIG. 2), contained a pair of BsmBI sites and a unique BsrGI allowing for easy screening of sgRNA-positive clones.

The pBK109 plasmid was modified further to include a pair of Sp1 binding sites. To this end, the plasmid was digested with KpnI- and PacI and cloned with a pair of the annealed and the phosphorylated oligonucleotides:

upper-
(SEQ ID NO: 5)
5′-TAATGGGCGGGACGTTAACGGGGCGGAACGGTAC-3′
lower-
(SEQ ID NO: 6)
5′-CGTTCCGCCCCGTTAACGTCCCGCCCATTAAT-3′

The resulting plasmid was named pBK176 (FIG. 1).

The following sgRNAs targeting eGFP oligonucleotides were introduced into pLentiCRISPRv2 and pBK109 creating pBK86 and pBK189, respectively:

(1) upper:
(SEQ ID NO: 7)
5′-CACCGGGGCGAGGAGCTGTTCACCG-3′
lower:
(SEQ ID NO: 8)
5′-AAACCGGTGAACAGCTCCTCGCCCC-3′
(2) upper:
(SEQ ID NO: 9)
5′-CACCGGGAGCGCACCATCTTCTTCA-3′
(3) upper:
(SEQ ID NO: 10)
5′-CACCGGGTGAACCGCATCGAGCTGA-3′
lower:
(SEQ ID NO: 11)
5′-AAACTGAGCTCGATGCGGTTCACCC-3′

PBK189, harboring GFP-sgRNA1, was further modified to include two copies of Sp1-binding motif. To this end, the plasmid was digested with NdeI and cloned with NdeI-NdeI fragment of pBK179. The resulting plasmid was named pBK198. To introduce sgRNAs targeting GABAA receptor α-2 into the expression cassette, the following oligonucleotides were used:

upper-
(SEQ ID NO: 12)
5′-CACCGTAATCGGCTTAGACCAGGAC-3′
lower-
(SEQ ID NO: 13
5′-AAACGTCCTGGTCTAAGCCGATTAC-3′

To amplify eGFP target region, the following primers were employed:

F-
(SEQ ID NO: 14)
5′-CAAGTCTCCACCCCATTGACG-3′
R-
(SEQ ID NO: 15)
5′-GAACTCCAGCAGGACCATGT-3′

To amplify off-target sequences of Sin3B, MTRF1L, NARF, INSC-1, BRAF-1 genes the following primers were employed:

for Sin3B—

F-
(SEQ ID NO: 16)
5′-TCCCTTTGGTCCTCTTGTTG-3′
R-
(SEQ ID NO: 17)
5′-CGCCCATCTCTGCTCTCTAC-3′

for MTRF1L—

F-
(SEQ ID NO: 18)
5′-ATGCTACTGAGGACCCCATC-3′
R-
(SEQ ID NO: 19)
5′-GCAGCCTTGCTTTTCTGTCT-3′

for NARF—

F-
(SEQ ID NO: 20)
5′-GGAGGCTGAGGTAGGAGGAT-3′
R-
(SEQ ID NO: 21)
5′-CTGGGACTATAGGCGCTCAC-3′

for INSC-1—

F-
(SEQ ID NO: 22)
5′-TCTGGTGGAGTTTGCTGTTG-3′
R-
(SEQ ID NO: 23)
5′-CCAGCTCATGAGGTTGTTGA-3′

for BRAF-1—

F-
(SEQ ID NO: 24)
5′-CTGAGGACGGAGGAGACAAG-3′
R-
(SEQ ID NO: 25)
5′-CGGGAGAGGAGAGAGGAAAT-3′

Vector Production:

Lentiviral vectors were generated using the transient transfection protocol, as described previously. Briefly, 15 μg vector plasmid, 10 psPAX2 packaging plasmid (Addgene, #12260), 5 μg pMD2.G envelope plasmid (Addgene #12259) and 2.5 μg pRSV-Rev plasmid (Addgene #12253) were introduced into 293T cells by transfection. To generate IDLV, pBK43 packaging cassette was employed (see above). Vector particles were collected from filtered conditioned medium at 72 h post-transfection. When necessary, the particles were purified using sucrose-gradient method, and concentrated over 100-fold by ultracentrifugation (2 h at 22000 rpm). Vector and viral stocks were aliquoted and stored at −80° C.

Tittering Vector Preps:

For GFP-containing integrase-competent viruses, the number of GFP-positive cells was counted, and the titer was calculated according to known methodology. For integrase-deficient and/or GFP-deficient vectors, the p²⁴gag ELISA was used, equating 1 ng p²⁴gag to 1×10⁴ particles. MOI was calculated as the ratio of the p²⁴gag-based estimation of viral particle number to target-cell number. P²⁴gag ELISA. The protocol was executed as per instructions in the HIV-1 p24 antigen capture assay kit (obtained from the NIH AIDS Vaccine Program). Briefly, high-binding 96-well plates (Costar) were coated with 100 μL monoclonal anti-p24 antibody obtained from the NIH AIDS Research and Reference Reagent Program (catalog #3537), which was diluted 1:1,500 in PBS. Coated plates were incubated at 4° C. overnight. Plates were blocked with 200 μL 1% BSA in PBS and washed three times with 200 μL 0.05% Tween 20 in cold PBS. Plates were incubated with 2004 samples, inactivated by 1% Triton X-100, for 1 h at 37° C. HIV-1 standards (catalog no. SP968F) were subjected to 2-fold serial dilution and added to the plates at a starting concentration equal to 4 ng/mL. Sample-diluent solution was RPMI 1640, supplemented with 0.2% Tween 20, 1% BSA. Samples were incubated at 4° C. overnight. Plates were then washed six times and incubated with 100 μL polyclonal rabbit anti-p24 antibody (catalog # SP451T), diluted 1:500 in RPMI 1640, 10% FBS, 0.25% BSA, and 2% normal mouse serum (NMS; Equitech-Bio), at 37° C. for 2 h. Plates were washed as above and incubated with goat anti-rabbit horseradish peroxidase IgG (Santa Cruz), diluted 1:10,000 in RPMI 1640 supplemented with 5% normal goat serum (NGS; Sigma), 2% NMS, 0.25% BSA, and 0.01% Tween 20 at 37° C. for 1 h. Plates were washed as above and incubated with TMB peroxidase substrate (KPL) at room temperature for 10 min. The reaction was stopped by adding 100 μL 1 N HCL. Plates were read by Microplate Reader at 450 nm and analyzed in Excel. The experiments were performed in duplicates.

Flow Cytometry:

HEK293T cells or HEK293T-eGFP cells were transduced with relevant vectors and examined for GFP fluorescence intensity. For FACS analysis, cells were harvested using 0.05% trypsin-EDTA solution. The samples were precipitated by centrifugation at 2000 rpm at 4° C., and the pellet was re-suspended in 1 mL cold PBS. An equal volume of 4% formaldehyde solution was added to the samples for 10 min. Samples were washed once in PBS and spun down by centrifugation. The pellet was re-suspended in 1 mL PBS. Samples were analyzed for GFP expression by the FACScan™ system (Becton Dickinson). Mean fluorescence intensity (MFI) and percentage of GFP-positive cells were determined. The experiments were executed in duplicates.

Western Blot:

Nucleus accumbens shell was micro-dissected from 300 μM-thick coronal brain slices. The collected tissue was incubated with RIPA buffer, (50 mM HEPES, pH 7.6, 1 mM EDTA, 0.7% DOC, 1% Nonidet P-40, 0.5 M LiCl). Total protein amounts were determined by Lowry assay using BSA as a standard. Lysates were mixed with 1× Red Loading Buffer (catalog no. 7723; Cell Signaling Technology), supplemented with 100 mM DTT, and denatured by boiling for 10 min. Subsequently, SDS polyacrylamide gel electrophoresis was performed followed by membrane transfer, which was then blocked by 5% nonfat dry milk for 60 min at room temperature with constant agitation. Anti-GABA (A) α2 Receptor antibody, #AGA-002 was acquired from Alomone Labs (Israel) and used at 1:250 dilution. The reference control antibody was mouse α-Tubulin (DM1A) antibody (Cell Signaling Technologies) used at 1:1000 dilution. The membrane was incubated with the antibody-containing solution for overnight at 4° C. through gentle agitation. The membrane was then washed three times for 5 min each, after which 0.05% Tween 20 in cold PBS (PBST) and the goat-anti-rabbit, or goat-anti-mouse secondary antibodies were applied at dilution 1:10000 for 1 h at room temperature or through gentle agitation. The blot detection was performed, using an enhanced chemiluminescence (ECL) detection system (Pierce).

Real-Time PCR:

To quantify rates of integration of IDLV and ICLV the following qPCR-protocol was used: Genomic DNA was isolated from the transduced cells according to standard methodology and digested with RNase A and DpnI overnight at 37° C. The following primers were used to amplify vector DNA:

RRE-F-
(SEQ ID NO: 26)
5′-GCAACAGACATACAAAC-3′
U6p-R-
(SEQ ID NO: 27)
AAAACTGCAAACTACCCAAGAAA-3′

β-Actin was used as a reference gene;

Actin-F-
(SEQ ID NO: 28)
5′-AATCTGCCACCACACCTTC-3′
Actin-R-
(SEQ ID NO: 29)
5′-GGGGTGTTGAAGGTCTCAAA-3′

ITaq™ Universal SYBR® Green Supermix was used for the reactions (Bio-Rad). Real-time PCR was executed using iCycler iQ System and the results were analyzed by iCycler software (Bio-Rad).

T7 Endonuclease I Assay:

Genomic DNA was isolated and PCR-amplified as described above. The PCR-products were extracted and purified from the gel using QIAGEN gel-extraction kit. 2 μL NEBuffer 2 and dH₂O were added for a total of 19 μL and subjected to denaturation-renaturation cycle in a PCR cycler as follows: 5 min, 95° C.; ramp down to 85° C. at −2° C./s; ramp down to 25° C. at −0.1° C./s; hold at 4° C. Next, T7 endo I enzyme (NEB) was added (1 μL (10 U)) to the reaction mix and the samples were incubated at 37° C. for 1 hour. Reaction was stopped by adding 2 μL of 0.25M EDTA and immediately loaded on a 1.2% agarose gel. The results were analyzed and quantified by E-Gel® Imager System software (Life Technologies).

In Vivo-Microinjections and Slice Electrophysiology.

All animal protocols were approved by the University of South Carolina Institutional Animal Care and Use Committee. Adult male Sprague-Dawley rats (300-350 g) were anesthetized with i.p. injections of a ketamine (80 mg/kg)/xylazine (12 mg/kg) mixture. IDLV-α2/Cas9 (2 μL) was injected bilaterally into the nucleus accumbens shell via a Neuros syringe (Hamilton) using the following stereotaxic coordinates (relative to bregma): 1.0 mm anterior, ±1.0 mm lateral, 5.0 mm ventral. At 35-47 days after the virus microinjections the rats were decapitated following isoflurane anesthesia. The brain was removed and coronal slices (300 μm) containing the nucleus accumbens shell were cut with a Vibratome (VT1000S, Leica Microsystems) in an ice-cold artificial cerebrospinal fluid solution (ACSF), in which NaCl was replaced by an equiosmolar concentration of sucrose. Control animals were treated similarly, but did not receive injection of the virus. ACSF consisted of 130 mM NaCl, 3 mM KCl, 1.25 mM NaH₂PO₄, 26 mM NaHCO₃, 10 mM glucose, 1 mM MgCl₂, and 2 mM CaCl₂ (pH7.2-7.4 when saturated with 95% O₂/5% CO₂). Slices were incubated in ACSF at 32-34° C. for 45 min and kept at 22-25° C. thereafter, until transfer to the recording chamber. Slices were viewed using infrared differential interference contrast optics under an upright microscope (Eclipse FN1, Nikon Instruments) with a 40× water-immersion objective. The recording chamber was continuously perfused (1-2 ml/min) with oxygenated ACSF heated to 32±1 1° C. using an automatic temperature controller (Warner Instruments). DL-AP5 (50 μM), DNQX (10 μM) were added to ACSF to block NMDA receptors and AMPA receptor, respectively. ACSF also contained TTX (0.5 μM) to block voltage-gated Na⁺ channels and isolate action-potential independent miniature inhibitory post-synaptic currents (mIPSCs). Recording pipettes were pulled from borosilicate glass capillaries (World Precision Instruments) to a resistance of 4-7MΩ when filled with the intracellular solution. The intracellular solution contained (in mM): 100 CsCH₃O₃S, 50 CsCl, 3 KCl, 0.2 BAPTA, 10 HEPES, 1 MgCl2, 2.5 phosphocreatine-2Na, 2 Mg-ATP, 0.25 GTP-Tris, adjusted to pH 7.2-7.3 (pH 7.2-7.3 with CsOH, osmolarity 280-290 mOsm). Medium spiny neurons in the nucleus accumbens shell were identified by their morphology and the low resting membrane potential (−70 to −85 mV). mIPSC recordings were obtained in whole-cell voltage-clamp mode (Vh=−70 mV) using a Multi-Clamp700B amplifier (Molecular Devices). Currents were low-pass filtered at 2 kHz and digitized at 20 kHz using a Digidata 1440A acquisition board and pClamp10 software (both from Molecular Devices). Access resistance (10-30MΩ) was monitored throughout the recordings by injection of 10 mV hyperpolarizing pulses and data were discarded if access resistance changed by >25% over the course of data acquisition. All analyses of intracellular recordings were carried out with Clampfit 10 (Molecular Devices). The time constant of decay was based on a double exponential fit to the decay phase of an average mIPSC trace computed from a minimum of 50 individual mIPSCs.

Whole-Exome Sequencing.

The off-target effects of IDLV-CRISPR/Cas9 and ICLV-CRISPR/Cas9 were assessed by whole-exome sequencing. To this end HEK293T cells were transduced with IDLV-gfp-sgRNA/Cas9 or and ICLV-gfp-sgRNA/Cas9 and harvested at day 21 pt. The respective gDNAs were hybridized to the probes of exome library (SeqCap EZ Library SR DNA-Seq; Roche). The library was pooled (4-plex) and exomes are enriched using Nimblegen protocol using SeqCap EZ Exome Enrichment Kit v3.0 (Roche). Each pool is sequenced in one IIlumina HiSeq lane V4 (125 bp Paired End) with on-target rates of ˜65%. The InDels were mapped using human genome build GRCh37-hg19 coordinates.

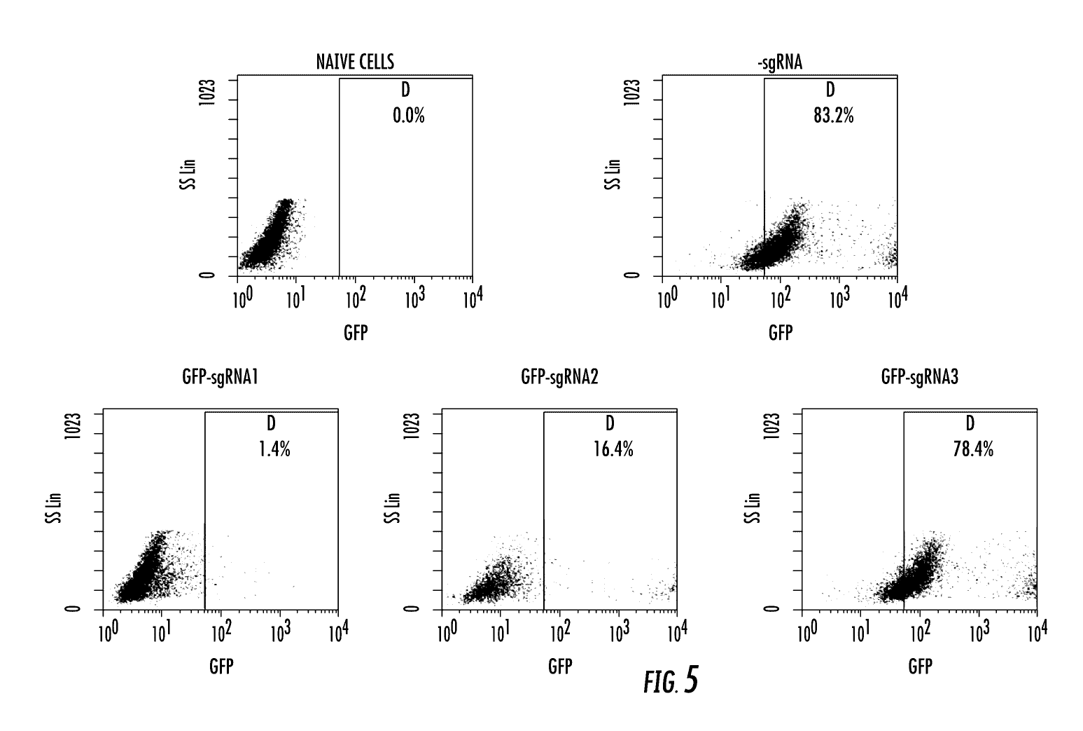

To test whether Sp1 can enhance the expression and production of integrating and non-integrating lentiviral vectors, a pair of Sp1-binding sites was cloned into the vector cassette upstream of the U6 promoter forming pBK176 (FIG. 1). To produce vesicular stomatitis virus G protein (VSV-G)-pseudotyped viral particles, vector plasmid with (pBK176) or without (pBK109) the Sp1 were cotransfected into 293T cells and packaged with integrase-competent type or integrase-deficient packaging cassettes to form vBK198 and vBK109, respectively. The production titers of resulting IDLV and ICLV were measured by p²⁴gag ELISA method (FIG. 3). The viral particles with (vBK198) or without (vBK109) Sp1 collected from the integrating vector were transduced into HEK293T cells and selected with puromycin to measure the overall functional production yield (FIG. 4). The p²⁴gag ELISA analysis of the vector stocks showed that the presence of Sp1 in the expression cassette results in an approximate fourfold increase in the production titers of both IDLV and ICLV (FIG. 3). Furthermore, the overall functional titers of the integrating and vector harboring Sp1 binding sites were increased by about sevenfold (FIG. 3).

A pLenti-CRISPR/Cas9 expression cassette was modified by removing 2-kbs of a buffer sequence and re-designing its polylinker site. These changes were introduced to reduce the size of the pLenti-CRISPR/Cas9 plasmid, and ease the cloning of sgRNA molecules. The new CRISPR/Cas9-expression cassette was found advantageous in terms of the recombination stability and the replication efficiency.

### Knock-Out Efficiency of IDLV-Based CRISPR/Cas9

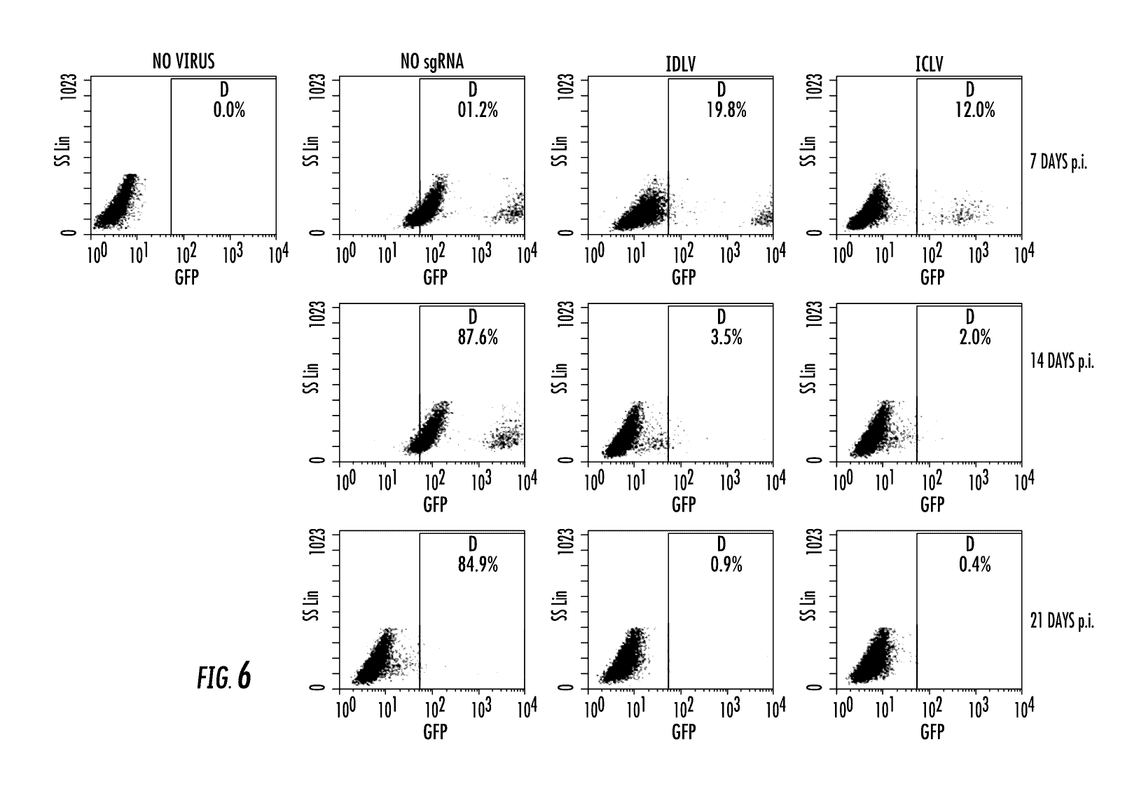

To examine whether the new vector system was capable of mediating an efficient gene knockout, three lentiviral vectors were designed targeting different parts of enhanced green fluorescent protein (eGFP) (sgRNA1, sgRNA2, sgRNA3). Reporter HEK293T cells constitutively expressing eGFP were transduced with the vectors and reductions in eGFP expression were evaluated by flow cytometry. Native cells and cells transduced with -sgRNA lentiviral vectors were used as control. The sgRNA1 vector demonstrated the strongest reduction of eGFP (FIG. 5) and was selected for further evaluation.

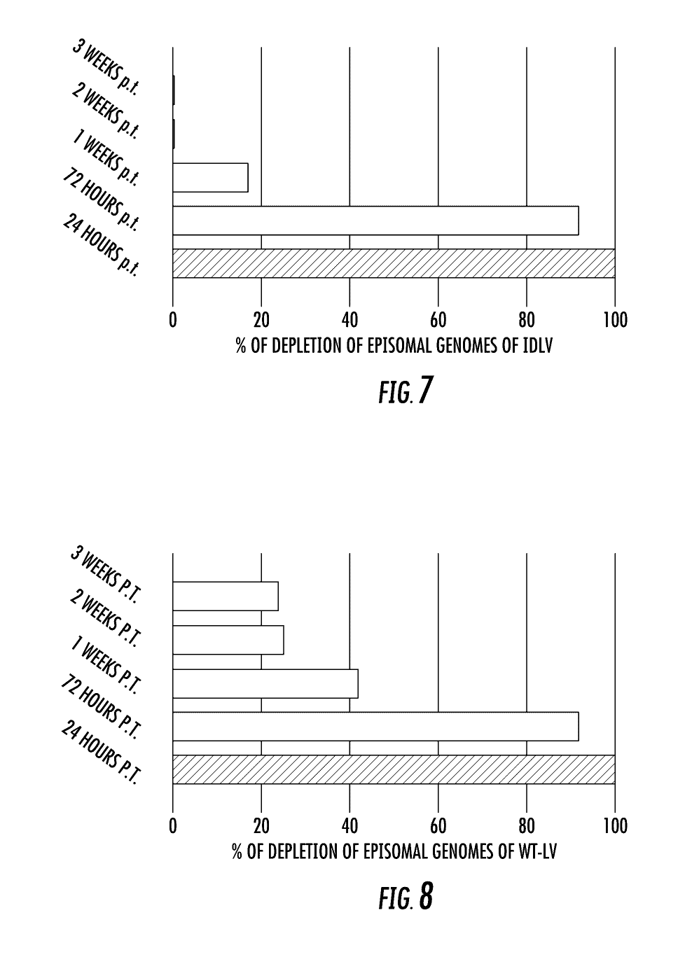

The efficiency of eGFP depletion between IDLV sgRNA1/Cas9 and ICLV sgRNA1/Cas9 vectors was compared. To this end, the vectors were transduced into HEK293T eGFP expressing cells and the knockout levels were evaluated at 7, 14, and 21-days post-transduction (pt). As shown in FIG. 6, both IDLV and ICLF vectors displayed an approximately fivefold reduction in the expression of eGFP as early as 7 days pt, with nearly a complete depletion of the signal by 21 days pt. Thus, IDLV-CRISPR/Cas9 platform is comparable to ICLV-CRISPR/Cas9 in terms of achieving efficient and sustained gene knockout in HEK293T cells.

The possibility that depletion of eGFP following lentivirus-mediated CRISPR/Cas9 transduction resulted from increased integration of CRISPR/Cas9 was addressed. To this end, the IDLV- and ICLV-transduced cells were cultured for up to three weeks to dilute out episomal genomes and subjected to the qPCR analysis to evaluate integration rates. As shown in FIG. 7, the levels of episomal genomes of IDLVs significantly decreased between 24 hours and 1 week pt and stabilized between two and three weeks pt. The overall frequency of IDLV integration measured at three weeks pt was about 1%. These results are in accord with previously published data showing similar rates of integrase-independent (illegitimate) integration of IDLVs, and thus suggest that IDLVs-CRISPR/Cas9 system was able to maintain its non-integrating status.

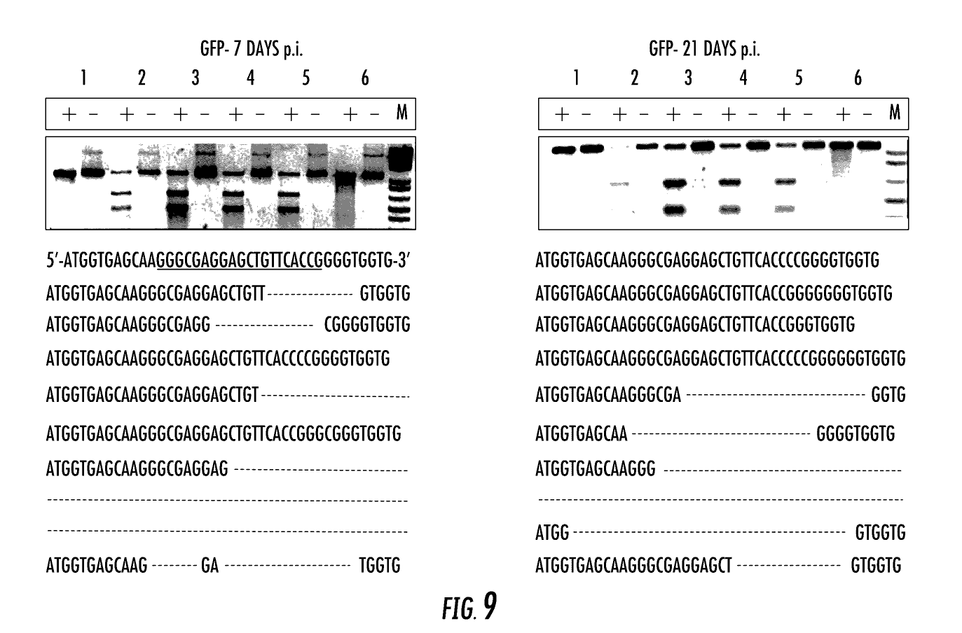

In contrast, the rate of ICLVs-CRISPR/Cas9 mediated integration was determined to be about 30% (FIG. 8), which is also in line with previously published data. Together, these findings demonstrate that while efficiency and sustainability of target gene knock-out is similar between ICLV- and IDLV-based CRISPR/Cas9 systems; IDLV-based CRISPR/Cas9 platform is associated with markedly lower integration rates.

On-Target Mutations Following Transduction with IDLV and ICLV

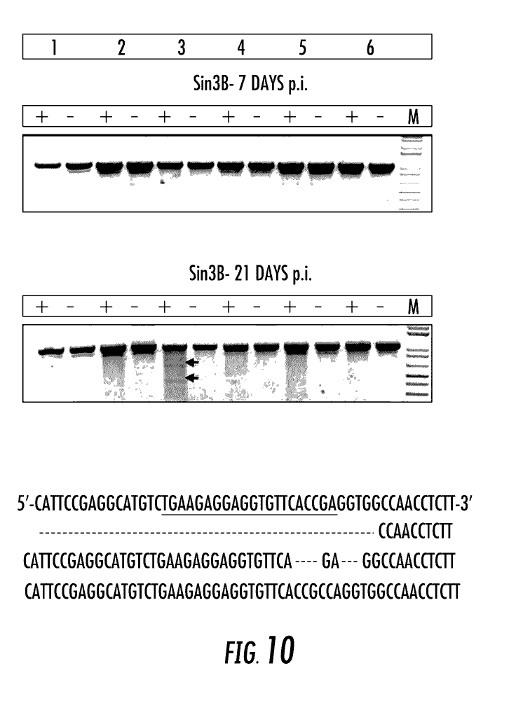

Having established the capacity of IDLV-CRISPR/Cas9 system to mediate a robust knockout of eGFP, target-specificity of non-integrating and integrating vectors was examined. To this end, a T7 endonuclease I assay was employed that detects heteroduplexes formed from annealing DNA stands following the double-strand cut induced by sgRNA/Cas9. The ability of CRISPR/Cas9 delivered by IDLV and ICLV to cleave on-target GFP sites was evaluated in the experimental setting described in FIG. 5 and FIG. 6. In agreement with the results shown in those figures, both ICLV and IDLV were able to efficiently induce InDels in the target sequence measured at day 7 pt (FIG. 9, top left). In FIG. 9 and FIG. 10, the gDNA isolated from the transduced cells was amplified with eGFP-specific primers and treated with T7 endo I (+) or left untreated (−).

Lanes were as follows:
1: naïve (untransduced cells);
2: ICLV-transduced cells at MOIs=1;
3: ICLV-transduced cells at MOIs=5;
4: IDLV-transduced cells at MOIs=1;
5: IDLV-transduced cells at MOIs=5 and
6: ICLV-transduced cells non-sgRNA-control.

Interestingly, neither increasing the vector concentration, nor extending the incubation time resulted in further upturn in the mutation rate (FIG. 9, top right). Mutations were not observed in naïve (untransduced) cells and following incubation with non-sgRNA-vectors (FIG. 9, lanes 1 and 6, respectively).

These results were confirmed by analyzing the samples with Sanger sequencing analysis. To this end, gDNA was extracted from ICLV and IDLV-transduced cells and amplified them with primers that flank the target eGFP sequence. The PCR products then were cloned into pCR2.1 TOPO vector and sequenced. Results are shown in FIG. 9, bottom left (ICLV) and right (IDLV). Cleavages were observed in the target sequences at rates of 84% and 80% for ICLV and IDLV, respectively and a random pattern of InDels formation. Thus, on-target activity was comparable between ICLV- and IDLV-based CRISPR/Cas9 systems.

Off-Target Mutations Following Transduction with IDLV and ICLV

To evaluate off-targeted activities of CRISPR/Cas9 delivered by IDLV and ICLV, five potential off-target sites were selected as predicted by CRISPR-Design Software (Massachusetts Institute of Technology). The sites were divided into three categories based on the level of homology to the target sequence: high, moderate and low, with two genes selected for each group. From the first set, a SIN3 transcription regulator family member B (Sin3B) gene was selected, showing a 3-bps mismatch outside the seed sequence, and a mitochondrial translational release factor 1-like (MTRF1-L) gene showing a 4-bps mismatch (1-inside and 4-outside the seed sequence). The ability of sgRNA/Cas9 to cleave within these regions was determined by T7 endonuclease I assay and Sanger sequencing analysis as described above.

As shown in FIG. 10, top, no InDel formation was detected in Sin3B gene at day 7 pt for either vector used at MOIs=1 and 5 (lanes were as described above for FIG. 9). The Sanger sequencing analysis confirmed these results (data not shown). However, at 3 weeks pt, a significant level of InDel formation was detected within Sin3B region for the ICLV used at MOI=5 (FIG. 10, middle). Following quantification, the InDel level was measured to be 5%. In contrast, no InDel formation was detected at 3 weeks pt in cells transduced with IDLV at a matching concentration (FIG. 10, middle).

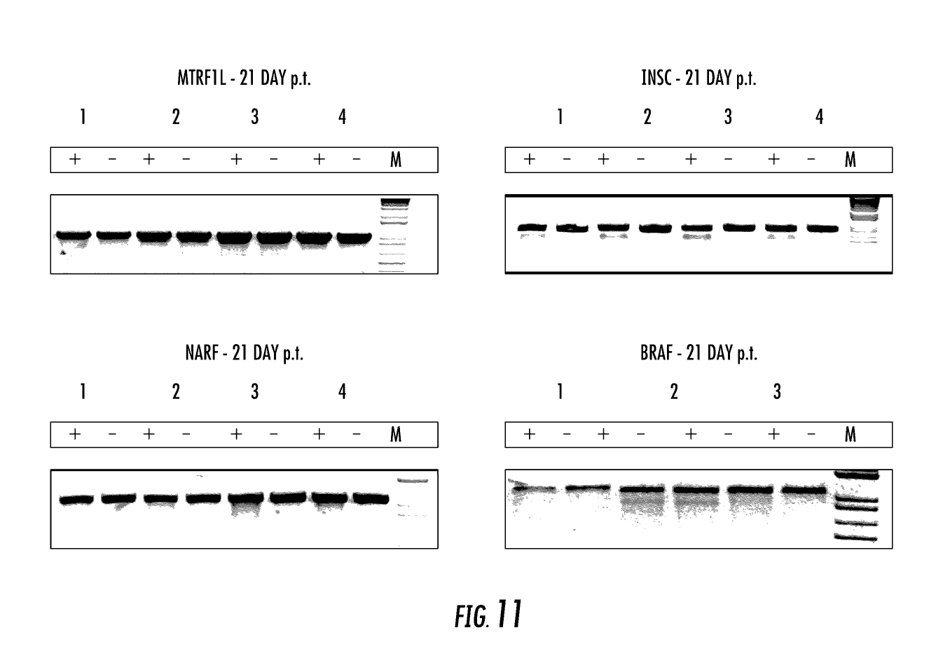

To further support these findings, ICLV-CRISPR/Cas9 derived PCR-products carrying the targeted Sin3B region were analyzed by Sanger sequencing. Randomly formed InDels were detected in four out of 50 clones (6%) in the region upstream to PAM, as shown in FIG. 10, bottom. However, no InDels were detected in cells transduced by IDLV. Furthermore, no ICLV- or IDLV-induced InDels were detected in a different highly homologous gene, MTRF1-L, when measured by either T7 endonuclease I assay (FIG. 11, top left) or Sanger sequencing (data not shown). Additionally, no InDels were detected in the moderate- and low-homology groups following transduction with the integrating CRISPR/Cas9 vector. In these groups, all PCR-products analyzed by Sanger sequencing at 3 weeks pt showed perfectly aligned setting and were not digested by T7 endonuclease I enzyme (FIG. 11).

To further evaluate the off-target capacity of IDLVs, a whole-exome sequencing (WES) analysis was carried out. To this end, 293T cells were transduced with IDLV-gfp-sgRNA/Cas9 or ICLV-gfp-sgRNA/Cas9 at MOIs=5. The cells were harvested at day 21 pt. gDNAs were isolated from the samples and hybridized to the probes of exome library (SeqCap EZ Library SR DNA-Seq; Roche). The library was pooled (4-plex) and exomes were enriched using Nimblegen protocol using SeqCap EZ Exome Enrichment Kit v3.0 (Roche). Each pool was sequenced in one IIlumina HiSeq lane V4 (125 bp Paired End) with on-target rates of about 65%. The InDels were mapped using human genome build GRCh37-hg19 coordinates.

The following criteria was applied to separate potential Cas9-induced DSBs from background DSBs. First, sequences with less than 40 total reads (×40) were not counted. Second, all known variants derived from dbSNPs were omitted. Third, a range of the InDels frequencies was defined as 1 to 25; higher rate was excluded as potential SNPs, lower rates were considered to be a background noise. Fourth, the nearest-neighbor sequences demonstrated high variability in InDels-formation were excluded from the database. Fifth, sequences were excluded that demonstrated low target-homology (70% or less at the seed region). Finally, DSBs were omitted in which PAM were not identified, or located 10 or more bps from the cleavage site.

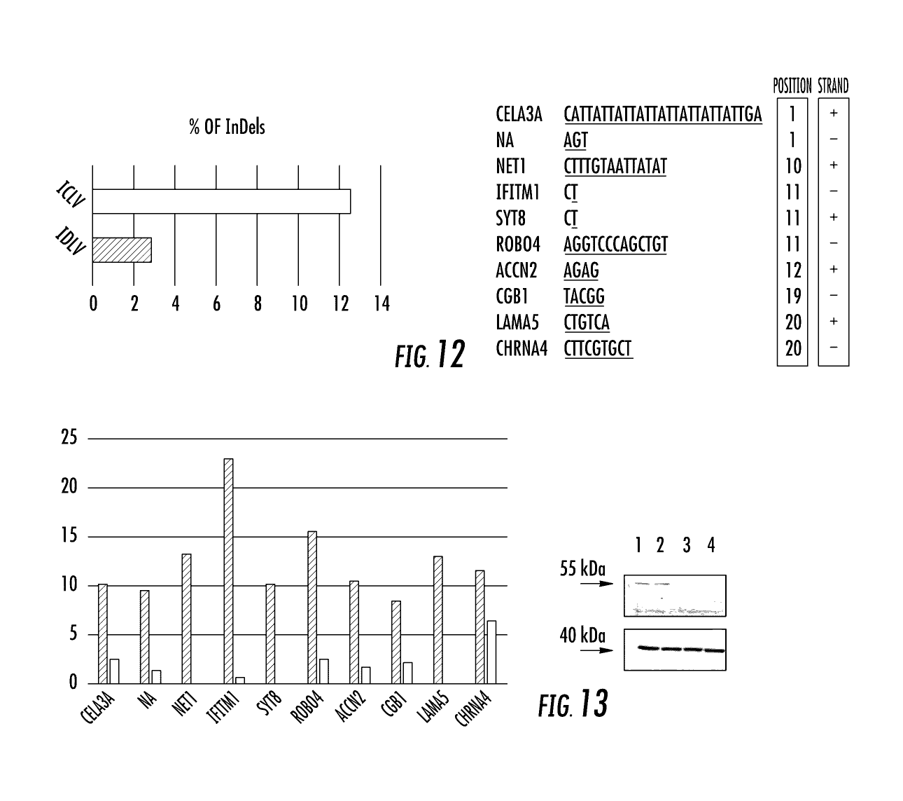

Applying these criteria, ten genes were identified in which ICLV-CRISPR/Cas9 had induced noticeable changes (FIG. 12). The frequencies of InDels at these sequences were detected to be in the range between 8.4 to 23 percent (FIG. 12 bottom left). In contrast, IDLV-CRISPR/Cas9 demonstrated significantly weaker capability to induce off-target InDels (FIG. 12 top left). Close-to-baseline frequency of InDels were measured in six genes; and a slight increase in three other genes (FIG. 12 top right). Nevertheless, higher rate of InDels was detected in CHRNA4 gene, suggesting that IDLV-CRISPR/Cas9 is capable of inducing off-target DNA mutations, whereas at significantly lower levels than its integrating counterpart.

ICLV-Mediated sqRNA/Cas9 Gene Editing In Vivo

The efficiency of the IDLV sgRNA/Cas9 platform was verified as an in vivo gene editing system. To do so, expression of a γ-amino-butyric acid A (GABA_{A}) receptor subunit α2 was targeted in the nucleus accumbens (NAc) of adult male Sprague-Dawley rats using an IDLV-α2/Cas9 vector. NAc is a region in the ventral striatum implicated in processing of reward and relevant for clinical symptoms of drug abuse and major depressive disorders. The majority of neurons (95%) within the NAc synthesize GABA and express GABA_{A} receptors that incorporate α1, α2, or α3 subunits when localized to synaptic membranes (Wisden et al., 1992; Ortinski et al., 2004a) of which α2 expression is the strongest (Pirker et al., 2000). The expression of α2 subunit was confirmed in NAc tissue homogenates from control animals and observed that 35-47 days following microinjection of the IDLV α2/Cas9 vector α2 protein declined to undetectable levels (FIG. 13).

The identity of the α subunit confers distinct functional properties on the assembled GABA_{A} receptor. Specifically, α2 and α3 subunit-containing GABA_{A} receptors generate currents that last longer than those generated by α1 subunits. This distinction was taken advantage of to verify IDLV α2/Cas9 efficiency at the level of receptor function and measured GABA_{A} receptor-mediated miniature inhibitory post-synaptic currents (mIPSCs) in medium spiny neurons of the NAc. At 35-47 days following microinjection of the IDLV α2/Cas9 duration of mIPSCs was characterized by broad cell-to-cell variability, contrasting sharply with the narrow distribution of mIPSC duration in cells from control animals (data not shown). Distribution of mIPSC amplitudes, an indicator of the number of post-synaptic receptors, however, was similar between cells from IDLV α2/Cas9-exposed and control slices (data not shown). These results indicate that IDLV α2/Cas9-induced knock-down of GABA_{A} receptor α2 subunit in the NAc, altered the subunit composition of post-synaptic GABA_{A} receptors in the NAc, but did not affect the number of receptors available for activation. Of greater relevance, these findings highlight the utility of IDLV sgRNA/Cas9 platform for long-term reduction of gene expression in non-dividing brain cells.

The episomal HIV-1 vectors were capable of attaining a strong and sustained CRISPR/Cas9 expression in post-mitotic neurons of the rat brain. Using and IDLV-based system, the efficient depletion of the GABA_{A} receptor α2 subunit protein in the nucleus accumbens shell was demonstrated. This depletion is associated with an increased variability of observed mIPSC decay times in the recorded neurons. The increased variability may be associated with altered contributions of short-lasting synaptic currents mediated by α1-containing GABA_{A} receptors and longer-lasting currents mediated by the α3-containing GABA_{A} receptors. Additionally, the IDLV-α2/Cas9 construct did not incorporate a fluorescent tag that could allow for positive identification of neurons transduced by the virus. Therefore, a population of cells that continued to express α2-containing GABA_{A} receptors may have contributed to these results.

It will be appreciated that the foregoing examples, given for purposes of illustration, are not to be construed as limiting the scope of this disclosure. Although only a few exemplary embodiments of the disclosed subject matter have been described in detail above, those skilled in the art will readily appreciate that many modifications are possible in the exemplary embodiments without materially departing from the novel teachings and advantages of this disclosure. Accordingly, all such modifications are intended to be included within the scope of this disclosure. Further, it is recognized that many embodiments may be conceived that do not achieve all of the advantages of some embodiments, yet the absence of a particular advantage shall not be construed to necessarily mean that such an embodiment is outside the scope of the present disclosure.

What is claimed is:

1. A viral vector comprising:
one or more structural components derived from a virus;
a nucleic acid sequence encoding a gene-editing molecule;
a promoter configured to initiate transcription of the nucleic acid sequence encoding the gene-editing molecule, wherein the promoter is free of introns;
an Sp1 transcription factor binding element and/or a NF-κB transcription factor binding element upstream of the promoter.

2. The viral vector of claim 1, wherein the vector is a retroviral vector.

3. The viral vector of claim 2, wherein the vector is a lentiviral vector.

4. The viral vector of claim 3, wherein the vector is an HIV-1 vector.

5. The viral vector of claim 1, wherein the gene-editing molecule comprises a gRNA.

6. The viral vector of claim 1, wherein the gene-editing molecule comprises a Cas9.

7. The viral vector of claim 1, wherein the viral vector is a CRISPR/Cas9 all-in-one plasmid viral vector.

8. The viral vector of claim 7, wherein the vector further comprises a second promoter, the second promoter being free of introns, the promoter of claim 1 being configured to initiate transcription of either the gRNA element or the Cas9 element of the binary plasmid viral vector, and the second promoter being configured to initiate transcription of the other of the gRNA element or the Cas9 element of the viral vector.

9. The viral vector of claim 8, wherein the viral vector further comprises a second Sp1 transcription factor binding element and/or a NF-κB transcription factor binding element upstream of the second promoter.

10. The viral vector of claim 1, the viral vector comprising multiple Sp1 transcription factor binding elements and/or multiple NF-κB transcription factor binding elements upstream of the promoter.

11. The viral vector of claim 1, wherein the Sp1 transcription factor binding element and/or the NF-κB transcription factor binding element is immediately upstream of the promoter.

12. The viral vector of claim 1, wherein the promoter comprises about 300 or fewer base pairs.

13. The viral vector of claim 1, wherein the promoter is a polymerase III promoter.

14. The viral vector of claim 1, wherein the promoter is a polymerase II promoter that has been modified to remove internal elements.

15. The viral vector of claim 1, wherein the viral vector in an integrating viral vector.

16. The viral vector of claim 1, wherein the viral vector is a non-integrating viral vector.

17. A delivery platform for a gene-editing transgene, the delivery platform comprising a vector plasmid in conjunction with one or more additional plasmids, the vector plasmid comprising the viral vector of claim 1.

18. A vector particle comprising the viral vector of claim 1.
